(19) United States
(12) Patent Application Publication
(10) Pub. No.: US 2010/0173918 A1
(43) Pub. Date: Jul. 8, 2010

(54) METHODS AND COMPOSITIONS FOR DETERMINING THE EFFICACY OF BREAST CANCER THERAPEUTICS

(75) Inventor: Sarah Bacus, Hinsdale, IL (US)

Correspondence Address: K&L Gates LLP, P.O. Box 1135, CHICAGO, IL, 60690, US

(73) Assignee: TARGETED MOLECULAR DIAGNOSTICS, LLC, Westmont, IL (US)

(21) Appl. No.: 12/529,115

(22) PCT Filed: Feb. 28, 2008
(86) PCT No.: PCT/US08/55276

§ 371 Date: Mar. 9, 2010

Related U.S. Application Data
(60) Provisional application No. 60/892,296, filed on Mar. 1, 2007.

(51) Int. Cl. A61K 31/517; G01N 33/53; G01N 33/574; A61P 35/04

(52) U.S. Cl. 514/266.24; 435/7.2; 435/7.23

(57) ABSTRACT

The present disclosure relates to a method, kit and controls for detecting phosphorylated Tyr¹²⁴⁸ in the c-erbB-2 protein as a predictor of breast cancer progression and as a predictor of therapeutic efficacy of drugs that inhibit both epidermal growth factor receptor and erbB2 protein kinases.

### BACKGROUND

Once a determination is made that a tissue or cell sample is malignant, the aggressiveness of the cancer and its clinical and biological characteristics must be determined so that an effective therapeutic approach can be developed to interfere with or eradicate the cancer.

A diagnosis can be confirmed through histological examination of a tissue or cell sample removed from a patient. Image analysis can be used, generally, to assess the affinity of stains for various biological markers. Examples of suitable affinity stains include chromagen-labeled monoclonal antibodies directed against the estrogen receptor (ER), the progesterone receptor (PR), the HER-2/neu protein, and the epidermal growth factor receptor (EGFR).

Affinity staining and image analysis has been used to facilitate the selection of optimal patient therapies for example, in the use of hormone therapy for cancers that are ER and PR positive and for anti-oncogene receptor therapy, such as using monoclonal antibodies directed against to HER-2/neu (Herceptin™), EGFR, or C225™, alone or in combination with chemotherapy. In addition, image analysis techniques can be used to quantitate other receptors such as those in the erbB receptor family (HER-1, HER-2/neu, HER-3, and HER-4), their ligands (EGF, NDF, and TGFα), and downstream signals (PI3 kinase, Akt, MAP kinase, and JUN kinase) (National Institute of Health Consensus Development Conference: Steroid Receptors in Breast Cancer, 1979, Vol. 2 No. 6; Kraus et al., 1989, Proc. Natl. Acad. Sci. U.S.A. 86:9193-97; Mendelsohn, 1990, Semin. Cancer Biol. 1:339-44; Hancock et al., 1991, Cancer Res. 51:4575-80; Peles et al., 1991, EMBO J. 10:2077-86; Peles et al., 1992, Cell 69:205-16; Arteaga et al., 1994, Cancer Res., 54:3758-65; Pietras et al., 1994, Oncogene 9:1829-38; Baselga et al., 1999, Proceedings of AACR NCI EORTC International Conference, Abstract 98; Cobleigh et al., 1999, J. Clin. Oncol. 17:2639-48; DiGiovanna, 1999, PPO Updates: Princ. Practice Oncol. 13:1-9; Shak, 1999, Semin. Oncol. 26:71-77; Sliwkowski et al., 1999, Semin. Oncol. 26:60-70; Vincent et al., 2000, Cancer Chemother. Pharmacol. 45: 231-38).

Certain protein tyrosine kinases are known to provide central switch mechanisms in cellular signal transduction pathways and are often involved in cellular processes such as cell proliferation, metabolism, survival and apoptosis. Several protein tyrosine kinases are known to be activated in cancer cells and to drive tumor growth and progression. Thus, interfering with tyrosine kinase activity provides an approach to cancer therapy especially in situations where affinity staining identifies their associated receptors in cancer cells. Therapeutic strategies include blocking kinase-substrate interaction, inhibiting the enzyme's adenosine triphosphate (ATP) binding site and blocking extracellular tyrosine kinase receptors on tumor cells, among others. Already several tyrosine kinase inhibitors (TKIs) have been approved as anti-cancer agents.

The erbB or HER family of transmembrane tyrosine kinase receptors, especially receptors erbB1 (or EGFR) and erbB2 (or Her2/neu), provides an important therapeutic target in a number of cancers. Her2/neu, for example, is overexpressed in about 20% to 30% of patients with aggressive breast cancer, while EGFR is overexpressed in several solid tumors. Consequently, new drugs have been developed that target these receptors individually or together and block the phosphorylation events that appear to trigger uncontrolled cancer cell growth.

Lapatinib ditosylate (GSK572016) is an epidermal growth factor receptor (EGFR) and ErbB-2 (Her2/neu) dual tyrosine kinase inhibitor under development for the treatment for certain solid tumors such as breast and lung cancer. The drug appears to arrest the development of breast cancer in some patients with metastatic, treatment-refractory disease. The drug could become an important new treatment option for breast cancer patients and potentially those with other difficult-to-treat solid tumors.

Thus, new methods are needed to identify whether a drug such as lapatinib that targets epidermal growth factor receptor (EGFR) and ErbB-2 (Her2/neu) expressing cancer cells is likely to be of therapeutic use. Such diagnostic methods and compositions could be used to identify circumstances in which drugs such as lapatinib are likely to be effective in treating a particular cancer.

### SUMMARY

New methods and compositions are disclosed for identifying cancers for which a drug that targets both an epidermal growth factor receptor and erbB2 are likely to be therapeutically effective. The method involves identifying phosphorylated Tyr¹²⁴⁸ in the peptide of amino acids from about 1242 through 1255 of c-erbB-2 in one or more cells (e.g., breast cancer cells) from a subject.

Methods of the present disclosure involve histochemical staining of a target epitope (e.g., phosphorylated Tyr¹²⁴⁸ in the peptide of amino acids from about 1242 through 1255 of c-erbB-2) in a biological sample. The staining protocol involves the use of one or more detectably labeled binding molecules that are known to bind to an epitope corresponding to phosphorylated tyrosine 1248 in a peptide substantially corresponding to the amino acid sequence in the range of from about 1242 to about 1255 in the c-terminal region of human c-erbB-2 protein. Typically, the binding molecule or molecules will be an antibody which could be either a monoclonal or polyclonal antibody. The stained sample is then viewed to determine the intensity of staining in target cells in an area of the sample. Visualization of a detectable stain provides an indication that the epitope is present.

In one method the level of the phosphorylated protein can be quantitated by determining the staining intensity in the cellular area. In one method the quantitation can include reference to staining intensities in a series of control cell pellets. Thus, the staining protocol can be carried out in a plurality of control cell pellets. The quantity of the epitope in each control cell pellet is independently known as by ELISA or other known methods. In addition, the level of the epitope in each of the control cell pellets is not the same such that a standard curve can be developed for reference. Then the staining intensity of the stained sample can be compared to the intensity in the standard curve as a measure of the quantity of the epitope.

The present disclosure provides methods for identifying an epitope in one or more cells of a biological sample by binding one or more detectably labeled binding molecules that selectively bind to an epitope corresponding to phosphorylated tyrosine 1248 in a peptide substantially corresponding to the amino acid sequence in the range of from about 1242 to about 1255 in the c-terminal region of human c-erbB-2 protein; detecting the binding molecule with a stain; viewing the staining in the biological sample in a cellular area that contains the cells to be examined in said biological sample; and determining whether the cells in the cellular area are stained and thereby indicate that the epitope is present.

In some embodiments, the methods further comprise determining the quantity of the target epitope by determining the staining intensity in the cellular area.

In some embodiments, the histochemical staining method is an immunohistochemical staining method.

In some embodiments, the methods further comprise determining the quantity of the target epitope by carrying out in a plurality of control cell pellets the histochemical staining method for the epitope, wherein the quantity of the epitope in each control cell pellet is independently known, and wherein the expression level of the epitope in each of the control cell pellets is not the same; determining the staining intensity in a defined representative cellular area for each of the stained control cell pellets; generating a calibration curve relating the known quantity of epitope with the average staining intensity in a defined cellular area for each of the control cell pellets; and determining the quantity of the epitope in the biological sample by comparing the intensity of the intensity of the stained target epitope in the cellular area to the calibration curve and deriving the quantity of the target protein from the calibration curve.

The present disclosure also provides methods for predicting the efficacy of epidermal growth factor receptor (EGFR) and ErbB-2 (Her2/neu) dual tyrosine kinase inhibitors comprising identifying an epitope in one or more cells of a biological sample by binding one or more detectably labeled binding molecules that selectively bind to an epitope corresponding to phosphorylated tyrosine 1248 in a peptide substantially corresponding to the amino acid sequence in the range of from about 1242 to about 1255 in the c-terminal region of human c-erbB-2 protein; detecting the binding molecule with a stain; viewing the staining in the biological sample in a cellular area that contains the cells to be examined in said biological sample; determining whether the cells in the cellular area are stained and thereby indicate that treatment of a tumor containing cells of the type within the cellular area with an epidermal growth factor receptor (EGFR) and ErbB-2 (Her2/neu) dual tyrosine kinase inhibitor would be efficacious.

The present disclosure provides methods for predicting the responsiveness of a subject to one or more epidermal growth factor receptor (EGFR) and ErbB-2 (Her2/neu) dual tyrosine kinase inhibitors by identifying an epitope corresponding to phosphorylated tyrosine 1248 in a peptide substantially corresponding to the amino acid sequence in the range of from about 1242 to about 1255 in the c-terminal region of human c-erbB-2 protein in one or more cells, wherein the identification of the epitope corresponding to phosphorylated tyrosine 1248 in human c-erbB-2 in one or more cells from the subject indicates that the subject is responsive to one or more epidermal growth factor receptor (EGFR) and ErbB-2 (Her2/neu) dual tyrosine kinase inhibitors.

In some embodiments, the inhibitor is lapatinib.

In some embodiments, the methods further comprise determining the quantity of the target epitope by determining the staining intensity in the cellular area.

In some embodiments, the methods further comprise determining the quantity of the target epitope by carrying out in a plurality of control cell pellets a histochemical staining method for the epitope using a detectably labeled binding molecule that is specific for said epitope, wherein the quantity of the epitope in each control cell pellet is independently known, and wherein the expression level of the epitope in each of the control cell pellets is not the same, determining the staining intensity in a defined representative cellular area for each of the stained control cell pellets; generating a calibration curve relating the known quantity of epitope with the average staining intensity in a defined cellular area for each of the control cell pellets; and determining the quantity of the epitope in the biological sample by comparing the intensity of the intensity of the stained target epitope in the cellular area to the calibration curve and deriving the quantity of the target protein from the calibration curve.

In some embodiments, the methods further comprise automated image analysis of the stained biological sample cells and control cells.

The present disclosure provides methods of treating cancer in a subject by obtaining a biological sample from a subject; binding one or more detectably labeled binding molecules to the biological sample, wherein the binding molecules selectively bind to an epitope corresponding to phosphorylated tyrosine 1248 in a peptide substantially corresponding to the amino acid sequence in the range of from about 1242 to about 1255 in the c-terminal region of human c-erbB-2 protein; detecting the presence of the epitope in the biological sample; and treating the subject with one or more epidermal growth factor receptor (EGFR) and ErbB-2 (Her2/neu) dual tyrosine kinase inhibitors.

The present disclosure also provides a kit for predicting the efficacy of epidermal growth factor receptor (EGFR) and ErbB-2 (Her2/neu) dual tyrosine kinase inhibitors comprising: one or more binding molecules that bind to phosphorylated tyrosine 1248 in a peptide substantially corresponding to the amino acid sequence in the range of from about 1242 to about 1255 in the c-terminal region of human c-erbB-2 protein; a plurality of control cell pellets containing the epitope, wherein the quantity of the epitope in each control cell pellet is independently known, and wherein the expression level of the epitope in each of the control cell pellets is not the same; and instructions for carrying out a histochemical staining method on said biological sample.

In some embodiments, the binding molecule is an antibody preparation. In some embodiments, the binding molecule is a monoclonal antibody preparation.

In some embodiments the kit further comprises a fixing solution. In some embodiments, the fixing solution comprises at least one phosphatase inhibitor.

In some embodiments, the biological sample comprises one or more cells. In some embodiments, the cells are from a solid tumor. In some embodiments, the solid tumor is breast or lung cancer.

In some embodiments, the methods may further comprise identifying a phosphorylated form of human c-erbB-3 protein.

Additional features and advantages are described herein, and will be apparent from, the following Detailed Description and FIGURE.

### DESCRIPTION OF THE FIGURES

FIG. 1 is a picture demonstrating the effect of phosphatase inhibitors on the binding assay.

### DETAILED DESCRIPTION

Generally, the present disclosure encompasses one or more binding molecules for detecting a particular epitope (e.g., phosphorylated tyrosine¹²⁴⁸ in a c-erbB-2 protein) thought to be predictive for clinical efficacy of drugs that target both an epidermal growth factor receptor and erbB2, such as lapatinib. In an embodiment the disclosure includes compositions which include one or more binding molecules and corresponding methods for identifying one or more cells (e.g., breast cancer cells) that contain phosphorylated Tyr¹²⁴⁸ in the peptide of amino acids from about 1242 through 1255 of c-erbB-2. The disclosure can include controls which can be used to determine the amount of the epitope in sample cells and methods for detecting and quantitating the amount of the epitope in cells treated by the disclosed methods. Further, the disclosure can include methods for analyzing images produced by the disclosed methods.

The present disclosure is based on the discovery that a highly predictive prognostic indicator for the efficacy of drugs that target both an epidermal growth factor receptor and erbB2, such as lapatinib, is the existence and quantity of phosphorylated tyrosine¹²⁴⁸ in the c-erbB-2 protein in target cancer cells (e.g., breast cancer cells). Tumor cells that have large quantities of this epitope are thought to be highly susceptible to treatment by drugs such as lapatinib. However, the disclosure is not limited to use as a prognostic indicator for lapatinib treatment. Rather, it can be used as an indicator for treatment by any therapeutic shown to be effective against cells that express this epitope. More generally, the disclosed compositions and methods can be used in any application in which knowledge of the level of this epitope is desired, for example in following treatment efficacy and patient prognosis before during or after cancer treatments or in research settings when it is desirable to determine the existence and or quantity of this epitope in an unknown sample.

The disclosure encompasses one or more binding molecules which can be used to detect the epitope. Any suitable binding molecule or molecules that are stable under the assay conditions and is suitably selective for the epitope in the phosphorylated form can be used. A binding molecule or molecules are suitably selective for the epitope when its binding is increased to a point where it can be detected as compared to its binding to the same amino acid sequence containing an unphosphorylated tyrosine. Antibodies are generally envisioned for use in the present disclosure however, other molecules such as binding molecules derived from antibodies (e.g., a Fab, Fab′, Fab′-SH, Fv, scFv, F(ab′)2 or a diabody), aptamers, peptamers or molecules that include them could also be used so long as they have the required stability and binding specificity. More specifically, one suitable antibody is a mouse monoclonal antibody known as c-erbB-2/HER-2/neu (Phospho-specific) Ab-18 (Clone PN2A) which is hypothesized to bind to an epitope corresponding to amino acids 1242-1255 of c-erbB-2 having a phosphorylated Tyr¹²⁴⁸.

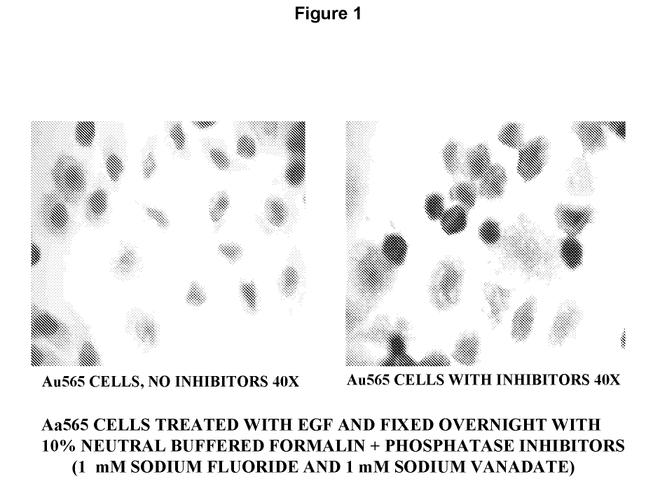

The present disclosure also includes methods for fixing cells and tissue samples for analysis. Generally, neutral buffered formalin is used. Any concentration of neutral buffered formalin that can fix tissue or cell samples without disrupting the epitope can be used. In one embodiment a solution of about 10 percent is used. Preferably, the method includes suitable amounts of phosphatase inhibitors to inhibit the action of phosphatases and preserve phosphorylation. Any suitable concentration of phosphatase inhibitor can be used so long as the biopsy sample is stable and phosphatases are inhibited, for example 1 mM NaF and/or Na₃VO₄ can be used. FIG. 1 is a picture of the effect of the phosphatase inhibitors on the binding assay. In the absence of the inhibitors no signal was observed.

In one method a tissue sample or tumor biopsy is removed from a patient and immediately immersed in a fixative solution which can and preferably does contain one or more phosphatase inhibitors, such as NaF and/or Na₃VO₄. Preferably, when sodium orthovanadate is used it is used in an activated or depolymerized form to optimize its activity. Depolymerization can be accomplished by raising the pH of its solution to about 10 and boiling for about 10 minutes. The phosphatase inhibitors can be dissolved in the fixative just prior to use in order to preserve their activity.

Fixed samples can then be stored for several days or processed immediately. To process the samples into paraffin after fixing, the fixative can be thoroughly rinsed away from the cells by flushing the tissue with water. The sample can be processed to paraffin according to normal histology protocols which can include the use of reagent grade ethanol. Samples can be stored in 70% ethanol until processed into paraffin blocks. Once samples are processed into paraffin blocks they can be analyzed histochemically for virtually any antigen that is stable to the fixing process.

The present disclosure provides methods to predict the efficacy of epidermal growth factor receptor (EGFR) and ErbB-2 (Her2/neu) dual tyrosine kinase inhibitors in a subject by identifying an epitope corresponding to phosphorylated tyrosine 1248 in a peptide substantially corresponding to the amino acid sequence in the range of from about 1242 to about 1255 in the c-terminal region of human c-erbB-2 protein in one or more cells from the subject, wherein the identification of the epitope corresponding to phosphorylated tyrosine 1248 in human c-erbB-2 in one or more cells from the subject indicates that the subject is responsive to one or more epidermal growth factor receptor (EGFR) and ErbB-2 (Her2/neu) dual tyrosine kinase inhibitors. Additionally, the methods may further comprise detecting the presence of phosphorylated c-erbB-3, wherein the identification of phosphorylated c-erbB-2 and c-erbB-3 indicate that the subject is responsive to one or more epidermal growth factor receptor (EGFR) and ErbB-2 (Her2/neu) dual tyrosine kinase inhibitors.

The present disclosure also provides methods of treating cancer (e.g., breast cancer) in a subject by obtaining a biological sample from a subject; binding one or more detectably labeled binding molecules to the biological sample, wherein the binding molecules selectively bind to an epitope corresponding to phosphorylated tyrosine 1248 in a peptide substantially corresponding to the amino acid sequence in the range of from about 1242 to about 1255 in the c-terminal region of human c-erbB-2 protein; detecting the presence of the epitope in the biological sample; and treating the subject with one or more epidermal growth factor receptor (EGFR) and ErbB-2 (Her2/neu) dual tyrosine kinase inhibitors (e.g., lapatinib). Additionally, the methods may further comprise detecting the presence of phosphorylated c-erbB-3, wherein the identification of phosphorylated c-erbB-2 and c-erbB-3 indicate that the subject is responsive to one or more epidermal growth factor receptor (EGFR) and ErbB-2 (Her2/neu) dual tyrosine kinase inhibitors.

The present disclosure also provides methods for identifying a particular epitope in a tissue or cell sample. Preferably, the method can also be used to determine the quantity of that epitope in the cells. Quantitation of a target epitope in a tissue or cell sample can utilize image analysis. Specifically, in a method expression levels of a target in a tissue or cell sample can be determined using a computer-aided image analysis system to enhance and process optical images of an histochemically stained tissue or cell sample, and to determine the optical density of the stained tissue or cell sample.

The quantity of a target in a sample can be determined histochemically by staining cells in the sample and staining a series of at least two control cell pellets using one or more detectably-labeled binding molecules, such as an antibody that selectively binds the target. The expression level of the target in the control cell pellets is known (for example, after determination by methods known in the art such as, inter alia, ELISA), and is not the same in the two samples. The optical density of the stained sample and stained control cell pellets can be determined and a calibration curve generated using the optical density of the control cell pellets. The expression level of the target protein in the sample cells can then be determined using the calibration curve. In preferred embodiments, the detectable label is a chromagen or fluorophore.

In a method control cells having varying levels of target can be generated by growth under varying conditions. Any cell that is capable of generating the phosphorylated tyrosine¹²⁴⁸ in the c-erbB-2 protein can be used, for example Au-565 cells. Cells containing low to intermediate levels of the epitope are grown in media containing 1% fetal bovine serum. Cells containing high levels of the epitope can be generated by growing them in media containing 1% fetal bovine serum and then incubating them with about 100 ng/ml EGF for about fifteen minutes. Cells containing low to no levels of the epitope are grown in the presence of media containing 1% fetal bovine serum and then treating them with 30 μM GW2974, a receptor antagonist for four hours. Following growth, cells can be harvested by standard methods and their epitope levels quantitated by standard methods, for example, such as ELISA. These cells can be used as control cell pellets that contain known quantities of the epitope.

In preferred embodiments, target protein-specific staining is detected, measured and quantitated automatically using automated image analysis equipment. Such equipment can include a light or fluorescence microscope, and image-transmitting camera and a view screen, most preferably also comprising a computer that can be used to direct the operation of the device and store and manipulate the information collected, most preferably in the form of optical density of certain regions of a stained tissue preparation. Image analysis devices useful in the practice of this disclosure include but are not limited to the CAS 200 (Becton Dickenson, Mountain View, Calif.), Chromavision or Tripath systems. Using such equipment the quantity of the target epitope in unknown cell samples can be determined using any of a variety of methods that are known in the art. The cell pellets can be analyzed by eye such that the optical density reading of the control cells can be correlated to a manual score such as 0, 1+, 2+ or 3+, as in Table 1 below which shows the correlation between quantitative image analysis data measured in optical density (OD) and manual score.

TABLE 1

| | |
|---|---|
| OD | Manual Score |

Phospho-HER2

| | |
|---|---|
| ≦5 | 0 |
| ≧5 to ≦17 | 1+ |
| ≧17 to ≦24 | 2+ |
| ≧24 | 3+ |

HER2

| | |
|---|---|
| ≦5 | 0 |
| ≧5 to ≦10 | 1+ |
| ≧10 to ≦22 | 2+ |
| ≧24 | 3+ |

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

### Immunohistochemical Staining Technique for Cells Embedded in Paraffin

### Preliminary Treatment of a Sample

One or more samples are treated to deparaffinize and hydrate the sections. For example, the sections may be incubated in three washes of xylene for five minutes each, two washes of 100% ethanol for ten minutes each, followed by two washes of 95% ethanol for ten minutes each. The sections are then washed twice in dH₂O for five minutes.

### Unmasking Antigen

Antigen is unmasked in either a Citrate/PBST or Citrate/TBST by bringing slides to a boil in 10 mM sodium citrate buffer (pH 6.0) then maintaining the slides at a sub-boiling temperature for ten minutes. The slides are then cooled at room temperature for thirty minutes. Alternatively, antigen unmasking can be performed with EDTA/PBST or EDTA/TBST by bringing slides to a boil in 1 mM EDTA pH 8.0 followed by fifteen minutes at a sub-boiling temperature. No cooling of the slides is necessary. Alternatively, slides can be brought to a boil in 10 mM Tris pH 10.0 followed by ten minutes at a sub boiling temperature. The slides may then be cooled on a bench top for thirty minutes. Alternatively, in a preferred embodiments, antigen unmasking can be performed with EDTA/PBST in a Decloaking Chamber (Biocare) with a SP1 of 125° C. for thirty seconds and a SP2 of 90° C. for ten minutes.

### Staining

Staining is carried out by washing sections in dH₂O three times for five minutes followed by treatment in 3% hydrogen peroxide for ten minutes. The sections are then washed in dH₂O twice for five minutes each and in wash buffer (Wash buffer, TBS (DakoCytomation)) for five minutes. Each section is washed with 100-400 μl blocking solution for one hour at room temperature. Blocking solution is then replaced by 100-400 μl diluted primary antibody (e.g., diluted in blocking solution). The sections are incubated with the antibody overnight at 4° C. After incubation, the antibody is removed and sections are washed in wash buffer three times for five minutes. About 100-400 μl secondary antibody (e.g., diluted in blocking solution per manufacture's recommendation), is added to each section followed by incubation for thirty minutes at room temperature.

ABC reagent for an ABC avidin stain is prepared according to the manufacturer's instructions and the solution is incubated for thirty minutes at room temperature. The secondary antibody solution is removed from the sections and the sections are washed three times with wash buffer for five minutes each. Approximately 100-400 μl ABC reagent is added to each section followed by incubation for thirty minutes at room temperature. The ABC reagent is removed and the sections are washed three times in wash buffer for five minutes. After the wash, 100-400 μl DAB or suitable substrate is added to each section and the staining is monitored. Upon development of color in the section, the slides are immersed in dH₂O. The slides can then be counterstained in mehatoxylin according to the manufacturer's instructions. The sections are then washed in dH₂O two times for five minutes.

Lastly, the sections are dehydrated by incubation in 95% ethanol two times for ten seconds each. This is repeated in 100% ethanol, incubating sections two times for ten seconds each and then with xylene twice for ten seconds. Coverslips may then mounted and examined for antigen.

### Example 2

### Expression of pHER-2 in Tumors Predicts a Favorable Response to Lapatinib Therapy

### Materials and Methods

Patients: Eligible adults (18 years of age) had histologically confirmed IBC (Inflammatory breast cancer) and a clinical diagnosis of IBC (e.g., presence of diffuse erythema and edema [peau d'orange], with or without an underlying palpable mass, involving the majority of the skin of the breast). Pathologic evidence of dermal lymphatic invasion was not required. Inclusion criteria included (a) locally advanced or metastatic disease that was refractory or had recurred after treatment with an anthracycline-containing regimen in the adjuvant or metastatic setting; (b) tumor—that was accessible for biopsy; (c) tumor that overexpressed HER-2 and/or expressed EGFR; (d) adequate renal, hepatic, bone marrow, and cardiac function; (e) an Eastern Cooperative Oncology (ECOG) performance status of 0 to 2; and (f) a life expectancy of at least 12 weeks. The number of prior chemotherapies, biologics (other than lapatinib), and antiestrogens was not restricted, with the last administration at least 4 weeks before study entry. EGF103009 was approved by the institutional review board from each participating institution, and written informed consent was obtained from all patients.

Study Design: EGF103009 was an open-label, two-stage, two-cohort, multicenter study to evaluate the activity and safety of lapatinib monotherapy in patients with IBC. A panel of protein biomarkers associated with tumor cell growth and survival was evaluated by semiquantitative immunohistochemistry (IHC) in fresh tumor biopsies collected within seven days of initiating lapatinib and processed as previously described (Spector N. L. et al., J Clin Oncol 23:2502-2512 (2005); Burns H. A. et. al., J Clin Oncol 23:5305-5313 (2005); Jones S. F. et al., J Clin Oncol 22:147s, 2004 (suppl. abstr 2083)).

Patients were assigned to cohort A if their tumor overexpressed HER-2 protein (2+ or 3+IHC) or exhibited gene amplification (positive on fluorescence in situ hybridization; ratio of HER-2:CEP17≧2) regardless of EGFR expression or to cohort B if their tumor expressed EGFR without HER-2 overexpression. Tumor biomarkers were analyzed in a blinded manner at a central Clinical Laboratory Improvement Amendments—certified College of American Pathology reference laboratory (Targeted Molecular Diagnostics, Westmont, Ill.) (Spector N. L., et al., J Clin Oncol 23:2502-2512 (2005)). Patients received lapatinib 1,500 mg once daily, continuously. The study design consisted of two stages; two responses were required in the first fifteen patients in either cohort before an additional fifteen patients were enrolled in that cohort (Green-Dahlberg design).

Patients underwent regular physical examinations and evaluations of performance status, body weight, CBC, serum chemistry, left ventricular ejection fraction by multiple gated acquisition scan or echocardiogram, and if applicable, digital photography to evaluate changes in chest wall/stun disease or assessment by imaging studies, computed tomography scan or magnetic resonance imaging.

Efficacy and Safety Evaluation: Clinical response was assessed at 4-week intervals and imaging performed at 8-week intervals until disease progression or withdrawal from study. Complete responses (CRs) or partial responses (PM) were confirmed at least 4 weeks later, Clinical responses were determined by treating physicians according to Response Evaluation Criteria in Solid Tumors (RECIST) where applicable (Themsse P. et al., J Natl Cancer Inst 92:205-216 (2000). Non-RECIST-measurable chest wall/skin disease, clinical responses were determined by the following criteria: (a) CR, disappearance of all disease that could be measured or evaluated; (b) PR, more than 50% decrease in extent of skin disease from baseline documented by digital photography; and (c) stable disease (SD), 20% to 49% decrease in extent of skin disease from baseline without the appearance of new lesions. Toxicity was graded according to the National Cancer Institute Common Terminology Criteria (NC1-CTCAE), version 3.0.

Immunohistochemical Analyses: Tumor biopsies were fixed in 10% neutral buffered formalin containing phosphatase inhibitors. Hematoxylin and eosin staining confirmed the presence of tumor. The EGFR pharmDx kit from Dako Cytomation (Carpinteria, Calif.) was used for EGFR IHC. The following antibodies were used: anti-HER-2 (1:80; Vector Labs, Burlingame, Calif.), -ER (1:100), -PR (1:200), -p53 (1:800), and -E-cadherin (1:300; Dako Cytomation), IGF-1R (1:200; Labvision/Neomarkers, Fremont, Calif.), -PTEN (1:400; Cascade Bioscience, Winchester, Mass.), -transforming growth factor a (TGF- or.; 1:40; Calbiochem, San Diego, Calif.), -heregulin (1:400) and -RhoC (1:50; Santa Cruz Biotechnologies, Santa Cruz, Calif.), phosphorylated (p) EGFR (1:25), pl-IER-2 (1:1200), -pHER-3 (1:125), and p-nuclear factor K B (pNFKB; Cell Signaling, Beverly, Mass.). Tissues were processed with antigen retrieval using EDTA buffer, pH 9.0 (Dako Cytomation) or with citrate buffer, pH 6.0 (Dako Cytomation), in the decloaker (Biocare Corporation, Concord, Calif.). All tissues were immunostained using the Autostainer (Dako Cytomation). Envision+dual-link polyper-horseradish peroxidase (HRP; Dako Cytomation) was used for all markers excluding RhoC, pEGFR, pHER-2, and pHER3. The ABComplex/HRP (Dako Cytomation) detected RhoC and the Mach3 kit (Biocare) detected pEGFR, pHER-2, and pHER-3. DAB+ was used for all markers except RhoC (DAB; Dako Cytomation). After immunostaining, all slides were counter-stained manually with methyl green (Dako Cytomation).

Statistical Analysis: The statistical focus of the study was to test the null hypothesis that the overall response rate (ORR) is no more than 15% versus the alternative hypothesis that this rate is at least 40%. Each cohort consisted of two stages, such that if there were fewer than two responses at the end of stage 1 (15 patients), then the cohort would be closed in favor of the null hypothesis. This provided power of more than 0.90 to correctly conclude that the treatment is effective (i.e., response rate of ≧40%), with an overall probability of falsely declaring the treatment effective if the response rate was 15% is less than 0.05 (type 1 error). Kaplan-Meier estimates of progression-free survival (PPS) were computed separately for cohort A and B. For this exploratory analysis, there were no statistical comparisons made between cohorts.

Baseline biomarkers β-catenin, bcl-2, ER, heregulin, p53, pEGFR, pHER-2, pHER3, PR, RhoC, TGF-α were classified as positive on the basis of IFIG values of 1+, 2+ or 3+(Spector N. L. et al., J Clin Oncol 23:2502-2512 (2005)). The remaining biomarkers, E-cadherin, IGFIR, and PTEN, were defined as positive when IHC values were 2+ or 3+. A multivariate analysis was used to evaluate the relationship between the 14 baseline biomarkers and response. For this, the ordinal level data for each biomarker (0, 1+, 2+, and 3+) was included in a partial least-squares discriminant analysis (SIMCA version 10.5.0, Umetrics Inc, Kinnelon, N.J.). Newly formed categoric variables are presented as counts (percentages). The two-sample Fisher's exact test was performed to assess the univariate relationship between baseline biomarker expression and patients' response status. All statistical calculations were two tailed, and statistical significance was set at the conventional 0.05 level. Statistical analyses were performed using SAS for Windows (version 8.2; SAS Institute, Cary, N.C.).

### Results

Forty-five patients who could be assigned to cohort A or B were recruited between March 2005 and February 2006 at 16 centers. Patient characteristics are summarized in Table 2. Dermal lymphatic invasion was present in the majority of tumor biopsies (73% in cohort A; 80% in cohort B). Histopathology was reviewed centrally, and invasive adenocarcinoma of the breast was confirmed. Protein analysis of fresh tumor biopsies exhibited a molecular profile consistent with that of IBC including (a) ER-negative (74%), (b) E-cadherin overexpression (80%), and (c) RhoC overexpression (100%). Patients in cohorts A and B received a median of four and three prior therapies, respectively (Table 2). All assessable patients had received at least one prior anthracycline-containing chemotherapy regimen, 86% and 80% of patients in cohorts A and B respectively had prior treatment with a taxane, and 50% (15 of 30) of the patients in cohort A received prior trastuzumab (Table 2). Two patients with confirmed IBC had recurrent metastatic disease without skin involvement at time of enrollment.

TABLE 2
Patient Characteristics

| | Cohort A | Cohort B |
|---|---|---|
| Characteristic | (n = 30) | (n = 15) |
| Age, years | | |
| Median | 54 | 54 |
| Range | 36-73 | 32-79 |
| Stage of disease, % | | |
| IIIB | 20 | 7 |
| IIIC | 13 | — |
| IV | 67 | 93 |
| Prior therapies | | |
| Median No. of regimens | 4 | 3 |
| Prior anthracycline, % | 100 | 100 |
| Prior taxane, % | 86 | 80 |
| Prior trastuzmab, % | 50 | 7 |
| Evidence of dermal invasion, % | 73 | 80 |

*Data not available for one patient in Cohort A.

The details of confirmed clinical responses are summarized in Table 3. Fifty percent (15 of 30) of patients with HER-2+ IBC (cohort A) experienced response. Of the 15 patients in cohort A who received prior trastuzumab therapy, six had response to lapatinib. Among the responders in cohort A were two patients (7%) whose best clinical response was a CR in the chest wall/skin or in RECIST-measurable lesions. An additional 13 patients (43%) had a PR in chest wall/skin and/or RECIST-measurable lesions as their best response. All responses in the skin occurred by the first assessment (week 4). Median duration of overall response (skin and RECIST) was 16.9 weeks (range, 8 to 31.7+weeks). The median PFS of cohort A patients was 14 weeks (95% CI, 15 to 32 weeks) with a median follow-up time of 15.3 weeks. In contrast, only one of 15 patients in cohort B had a clinical response (PR in skin/chest disease). Consequently, enrollment of patients into cohort B was closed. The median PFS of cohort B patients was 4 weeks (95% CI, 4 to 12 weeks) with a median follow-up of 4.1 weeks.

TABLE 3
Patient Responses

| | Skin | RECIST | Prior | Site(s) of RECIST |
|---|---|---|---|---|
| Cohort | Response | Response | Trastuzmab | Measurable Disease |
| A | CR | PR* | No | Breast, liver, skin |
| A | CR | SD | No | Breast, skin |
| A | PR | NA | No | Lymph node |
| A | PR | PR* | Yes | Breast, chest wall |
| A | PR | PR* | Yes | Chest wall |
| A | PR | PR | Yes | Lymph node |
| A | PR | PR* | No | Breast, chest wall, lymph |
| | | | | node |
| A | PR | PR | No | Breast, lymph node, skin |
| A | PR | PR | No | Breast |
| A | PR | SD | Yes | Breast, chest wall, lymph |
| | | | | node |
| A | PR | SD | Yes | Breast |
| A | PR | SD | Yes | Chest wall |
| A | PR | SD | No | Breast, bone, pleura |
| A | PR | SD | No | Breast, bone, pleura |
| A | PR | SD | No | Breast, pleura, skin |
| B | PR | SD | No | Breast, lymph node |

Abbreviations:
RECIST, Response Evaluation Criteria in Solid Tumors;
CR, complete response,
PR, partial response;
SD, stable disease,
NA, not assessable by RECIST.
*Confirmatory scan was performed before day 28 or is missing.

Lapatinib monotherapy was generally well tolerated in both cohorts. The most common adverse events included grade ½ diarrhea (49%), musculoskeletal pain (42%), and skin rash (36%). Serious adverse events (grades ¾) included pain (16%), dyspnea (11%), and diarrhea (11%; Table 4).

TABLE 4
Adverse Events Occurring in >5% (n > 3) of Patients (N = 45)

Total

| | |
|---|---|
| No. of | Grade > 3 |

| Event | Patients | % | No. | % |
|---|---|---|---|---|
| Laboratory | | | | |
| Elevated ALT/AST | 3 | 7 | 2 | 4 |
| Elevated alkaline phosphatase | 4 | 9 | 0 | 0 |
| Hyperbilirubinemia | 4 | 9 | 1 | 2 |
| Cardiovascular | | | | |
| Decreased LVEF* | 3 | 7 | 0 | 0 |
| Respiratory | | | | |
| Dyspnea | 9 | 20 | 5 | 11 |
| Pleural effusion | 5 | 11 | 4 | 9 |
| GI | | | | |
| Diarrhea | 27 | 60 | 5 | 11 |
| Constipation | 3 | 7 | 0 | 0 |
| Nausea | 11 | 24 | 0 | 0 |
| Vomiting | 9 | 20 | 0 | 0 |
| Skin/musculoskeletal | | | | |
| Rash+ | 16 | 36 | 0 | 0 |
| Pruritus | 5 | 11 | 0 | 0 |
| Pain± | 26 | 58 | 7 | 16 |
| Edema | 6 | 13 | 1 | 2 |
| Systemic | | | | |
| Infection | 6 | 13 | 2 | 4 |
| Anorexia | 10 | 22 | 1 | 2 |
| Fatigue | 11 | 24 | 0 | 0 |
| Anemia | 3 | 7 | 2 | 4 |

NOTE.
Adverse events were evaluated using the National Cancer Institute Common Toxicity Criteria version 2.0.
Abbreviation:
LVEF, left ventricular ejection fraction.
*Defined as >20% decrease in LVEF (as a percentage of baseline) that is asymptomatic and/or the ejection fraction is less than the institution's lower limit of normal.
+One report of rash was not graded.
±Mostly musculoskeletal pain (76% of cases).

Tumors in both cohorts exhibited similar biomarker profiles indicative of an IBC phenotype (i.e., RhoC and E-cadherin protein overexpression) (Colpaert C. G. et al., Br J Cancer 88:718-725 (2003); Van Golan K. L. et al., Cancer Res 60:5832-5838 (2000); Charafe-Jauffret E. et al., J Pathol 202:265-273 (2004)). Most tumors expressed the HER-family ligands TGF-α and heregulin; however, tumors from HER-2+ patients had increased expression of heregulin protein by IHC compared with tumors from HER-2− patients (P=0.012 by Wilcoxon statistic on IHC scores; data not shown) (Colpaert C. G. et al., Br J Cancer 88:718-725 (2003)). The baseline molecular profile (14 biomarkers) of tumors that responded to lapatinib versus those that did not in cohort A was explored by a multivariate analysis of the IHC scores (0 to 3+; Table 5). pHER-3 expression and lack of p53 expression were significantly associated with response (P<0.05 by multivariate analysis; P=0.021 and 0.033, respectively, by univariate analysis). Patients in cohort A whose tumors coexpressed pHER-2 and pHER-3 were more likely to respond (nine of 10 v four of 14; P=0.0045) than were patients whose tumors did not coexpress the phosphorylated receptors.

TABLE 5
Molecular Phenotype of Responding and Nonresponding
Patients in Cohort A

| | Respondents (n = 15) | | | Nonrespondents (n = 15) | | |
|---|---|---|---|---|---|---|
| | No. | Total | | No. | Total | |
| Phenotype | Affected | No. | % | Affected | No. | % |
| HER activation | | | | | | |
| status | | | | | | |
| pEGFR | 5 | 11 | 45 | 9 | 14 | 64 |
| pHER-2 | 11 | 12 | 92 | 13 | 14 | 93 |
| pHER-3 | 10 | 12 | 83 | 5* | 14 | 36 |
| ErbB ligands | | | | | | |
| Heregulin | 12 | 12 | 100 | 15 | 15 | 100 |
| TGF-\acute{α} | 12 | 12 | 100 | 14 | 14 | 100 |
| IBC phenotype | | | | | | |
| ER | 4 | 12 | 33 | 3 | 14 | 21 |
| PR | 3 | 12 | 25 | 2 | 14 | 14 |
| E-cadherin | 12 | 12 | 100 | 15 | 15 | 100 |
| IGF-1R | 10 | 12 | 83 | 13 | 15 | 87 |
| RhoC | 9 | 9 | 100 | 15 | 15 | 100 |
| Apoptosis and | | | | | | |
| tumor suppressors | | | | | | |
| pTEN deficient | 8 | 12 | 67 | 8 | 14 | 57 |
| p53 | 2 | 9 | 22 | 11+ | 15 | 73 |
| Bcl2 | 5 | 10 | 50 | 5 | 15 | 33 |
| β-catenin | 12 | 12 | 100 | 14 | 15 | 93 |

Abbreviations:
HER, human epidermal growth factor receptor;
p-, phosphorylated;
EGFR, epidermal growth factor receptor;
TGF, transforming growth factor;
IBC, inflammatory breast cancer;
ER, estrogen receptor;
PR, progesterone receptor;
IGF, insulin-like growth factor;
PTEN, phosphate and tensin homolog 10.
*P = .021
+P = .033

Bcl-2 and β-catenin expression did not correlate with response to lapatinib. PTEN deficiency (0 or 1+by IHC) has been associated with resistance to trastuzumab monotherapy but did not preclude response to lapatinib, because 67% of the responders were PTEN-deficient. In addition, coexpression of IGF-1R, which has also been associated with trastuzumab resistance occurred in 83% and 87% of responders and nonresponders, respectively, and did not affect the likelihood of response to lapatinib (Smith B. L. et al., Br J Cancer 91:1190-1194 (2004); Lu Y. et al., J Natl Cancer Inst 93:1852-1857 (2001)).

While the present disclosure has been described and illustrated herein by references to various specific materials, procedures and examples, it is understood that the disclosure is not restricted to the particular combinations of material and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method for identifying an epitope in one or more cells of a biological sample, the method comprising:
(a) binding one or more detectably labeled binding molecules that selectively bind to an epitope corresponding to phosphorylated tyrosine 1248 in a peptide substantially corresponding to the amino acid sequence in the range of from about 1242 to about 1255 in the c-terminal region of human c-erbB-2 protein;
(b) detecting the binding molecule with a stain;
(c) viewing the staining in the biological sample in a cellular area that contains the cells to be examined in said biological sample; and
(d) determining whether the cells in the cellular area are stained and thereby indicate that the epitope is present.

2. The method of claim 1, further comprising determining the quantity of the target epitope by determining the staining intensity in the cellular area.

3. The method of claim 1, wherein the histochemical staining method is an immunohistochemical staining method.

4. The method of claim 1, further comprising determining the quantity of the target epitope by
(a) carrying out in a plurality of control cell pellets the histochemical staining method for the epitope, wherein the quantity of the epitope in each control cell pellet is independently known, and wherein the expression level of the epitope in each of the control cell pellets is not the same;
(b) determining the staining intensity in a defined representative cellular area for each of the stained control cell pellets;
(c) generating a calibration curve relating the known quantity of epitope with the average staining intensity in a defined cellular area for each of the control cell pellets; and
(d) determining the quantity of the epitope in the biological sample by comparing the intensity of the intensity of the stained target epitope in the cellular area to the calibration curve and deriving the quantity of the target protein from the calibration curve.

5. A method for predicting the efficacy of epidermal growth factor receptor (EGFR) and ErbB-2 (Her2/neu) dual tyrosine kinase inhibitors, the method comprising identifying an epitope in one or more cells of a biological sample by:
(a) binding one or more detectably labeled binding molecules that selectively bind to an epitope corresponding to phosphorylated tyrosine 1248 in a peptide substantially corresponding to the amino acid sequence in the range of from about 1242 to about 1255 in the c-terminal region of human c-erbB-2 protein;
(b) detecting the binding molecule with a stain;
(c) viewing the staining in the biological sample in a cellular area that contains the cells to be examined in said biological sample;
(d) determining whether the cells in the cellular area are stained and thereby indicate that treatment of a tumor containing cells of the type within the cellular area with an epidermal growth factor receptor (EGFR) and ErbB-2 (Her2/neu) dual tyrosine kinase inhibitor would be efficacious.

6. A method for predicting the responsiveness of a subject to one or more epidermal growth factor receptor (EGFR) and ErbB-2 (Her2/neu) dual tyrosine kinase inhibitors, the method comprising: identifying an epitope corresponding to phosphorylated tyrosine 1248 in a peptide substantially corresponding to the amino acid sequence in the range of from about 1242 to about 1255 in the c-terminal region of human c-erbB-2 protein in one or more cells, wherein the identification of the epitope corresponding to phosphorylated tyrosine 1248 in human c-erbB-2 in one or more cells from the subject indicates that the subject is responsive to one or more epidermal growth factor receptor (EGFR) and ErbB-2 (Her2/neu) dual tyrosine kinase inhibitors.

7. The method of any one of claim 5 or 6, wherein the inhibitor is lapatinib.

8. The method of any one of claim 5 or 6, wherein the cells are breast cancer cells.

9. The method of claim 5, further comprising determining the quantity of the target epitope by determining the staining intensity in the cellular area.

10. The method of claim 5, further comprising determining the quantity of the target epitope by:
(a) carrying out in a plurality of control cell pellets a histochemical staining method for the epitope using a detectably labeled binding molecule that is specific for said epitope, wherein the quantity of the epitope in each control cell pellet is independently known, and wherein the expression level of the epitope in each of the control cell pellets is not the same,
(b) determining the staining intensity in a defined representative cellular area for each of the stained control cell pellets;
(c) generating a calibration curve relating the known quantity of epitope with the average staining intensity in a defined cellular area for each of the control cell pellets; and
(d) determining the quantity of the epitope in the biological sample by comparing the intensity of the intensity of the stained target epitope in the cellular area to the calibration curve and deriving the quantity of the target protein from the calibration curve.

11. The method for predicting the efficacy of epidermal growth factor receptor (EGFR) and ErbB-2 (Her2/neu) dual tyrosine kinase inhibitors of claim 5, further including automated image analysis of the stained biological sample cells and control cells.

12. A method of treating cancer in a subject, the method comprising:
(a) obtaining a biological sample from a subject;
(b) binding one or more detectably labeled binding molecules to the biological sample, wherein the binding molecules selectively bind to an epitope corresponding to phosphorylated tyrosine 1248 in a peptide substantially corresponding to the amino acid sequence in the range of from about 1242 to about 1255 in the c-terminal region of human c-erbB-2 protein;
(c) detecting the presence of the epitope in the biological sample; and
(d) treating the subject with one or more epidermal growth factor receptor (EGFR) and ErbB-2 (Her2/neu) dual tyrosine kinase inhibitors.

13. The method of claim 12, wherein the cancer is breast cancer.

14. The method of claim 12, wherein the biological sample comprises one or more cells.

15. The method of claim 12, wherein the binding molecules are antibodies.

16. The method of claim 15, wherein the antibodies are monoclonal antibodies.

17. The method of claim 12, wherein the epidermal growth factor receptor (EGFR) and ErbB-2 (Her2/neu) dual tyrosine kinase inhibitor is lapatinib.

18. A kit for predicting the efficacy of epidermal growth factor receptor (EGFR) and ErbB-2 (Her2/neu) dual tyrosine kinase inhibitors, the kit comprising: (a) one or more binding molecules that bind to phosphorylated tyrosine 1248 in a peptide substantially corresponding to the amino acid sequence in the range of from about 1242 to about 1255 in the c-terminal region of human c-erbB-2 protein;
(b) a plurality of control cell pellets containing the epitope, wherein the quantity of the epitope in each control cell pellet is independently known, and wherein the expression level of the epitope in each of the control cell pellets is not the same; and
(c) instructions for carrying out a histochemical staining method on said biological sample.

19. The kit of claim 18, wherein the binding molecule is an antibody preparation.

20. The kit of claim 18 wherein the binding molecule is a monoclonal antibody preparation.

21. The kit of claim 18 further comprising a fixing solution.

22. The kit of claim 18 further comprising a fixing solution containing at least one phosphatase inhibitor.
